(19) United States
(12) Plant Patent Application Publication
(10) Pub. No.: US 2002/0178475 P1
(43) Pub. Date: Nov. 28, 2002

(54) Chrysanthemum plant named 'MOZA LOT 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/865,150

(22) Filed: May 24, 2001

(51) Int. Cl. A01H 5/00

(52) U.S. Cl. PLT/291

(57) ABSTRACT

A new and distinct Chrysanthemum plant variety characterized by a daisy type inflorescence with pink/white color ray florets, consistent flowering response to short days, blooming consistently after 49 days of short day length, small foliage, short internode length, and consistent natural season flowering habit during late September.

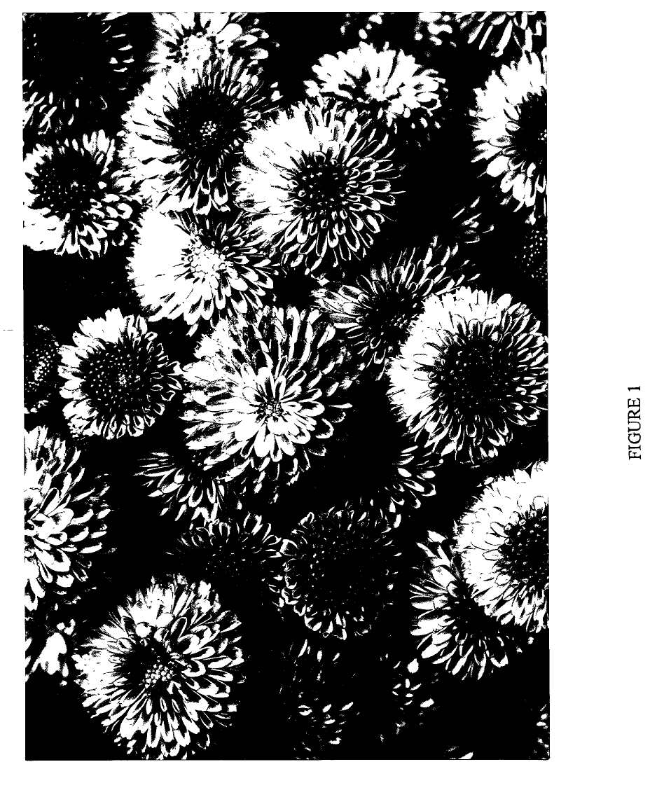

### BACKGROUND OF THE INVENTION

The present invention relates to a new and distinct cultivar of Chrysanthemum plant, botanically known as Dendranthema morifolium and herein after referred to by the cultivar name ‘MOZA LOT 01.’

This new cultivar originated from hybridization of a female parent Chrysanthemum cultivar (unpatented) and a male parent Chrysanthemum cultivar (unpatented in the United States). The new cultivar was discovered by the inventor in November 1998.

Asexual reproduction of the new cultivar by apical tip cutting and meristem tissue culture was performed in Oxnard, CA and has shown that the unique features of this new cultivar are stable and reproduced true to type on successive generations.

### SUMMARY OF THE INVENTION

The cultivar ‘MOZA LOT 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA LOT 01.’ These characteristics in combination distinguish ‘MOZA LOT 0’ as a new and distinct Chrysanthemum:

1. Daisy type inflorescence with pink/white color ray florets;

2. Consistent flowering response to short days, blooming consistently after 49 days of short day length;

3. Small foliage;

4. Short internode length; and

5. Consistent natural season flowering habit during late September.

Plants of the new cultivar are similar to plants of the female parent cultivar in most horticultural characteristics, however plants of the new cultivar differed from plants of the female parent cultivar in the following characteristics:

1. The new cultivar has different color ray florets;

2. The new cultivar is taller; and

3. The new cultivar has a more flexible plant shape.

Plants of the new cultivar are similar to plants of the male parent cultivar in most horticultural characteristics, however plants of the new cultivar differed from plants of the male parent cultivar in the following characteristics:

1. The new cultivar has different color ray florets;

2. The new cultivar is taller; and

3. The new cultivar naturally blooms 2 weeks later.

### BRIEF DESCRIPTION OF THE PHOTOGRAPH

The photograph shows a close up view of the flowers and buds of the new cultivar.

### DETAILED BONTICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types. Botanical classification: Dendranthema morifolium cultivar ‘MOZA LOT 01’ Commercial classification: Garden-type Chrysanthemum Parentage: Hybridization of female parent Chrysanthemum cultivar (unpatented in the United States) with a male parent Chrysanthemum cultivar (unpatented in the United States)

Propagation

Time to rooting.—7 to 14 days at approximately 21° C.

Rooting habit.—Fine, fibrous

Plant

Growth habit.—Mounding herbaceous perennial

Height.—Approximately 22 cm

Growth rate.—Moderate

Branching characteristics.—Moderate Branching

Length of lateral branches.—Approximately 16 cm

Number of leaves per lateral branch: Approximately 14

Stem Color.—Near 138A

Foliage

Leaf.—Arrangement: Alternate single Average Length: Approximately 6.1 cm Average Width: Approximately 3.4 cm Shape of blade: Ovate Apex: Acuminate Base: Attenuate Attachment: Stalked Margin: Serrate Texture of top surface: Lightly pubescent Texture of bottom surface: Lightly pubescent Color: young foliage upper side: Near 138A young foliage under side: Near 138B mature foliage upper side: Near 138A mature foliage underside: Near 138B venation upper side: Near 138C venation under side: Near 137C

Petiole.—Average Length: Approximately 2.0 cm

Bloom

Inflorescence.—Flowering habit: Induced by darkness period greater than 13.5 hours, approximately 49 days of appropriate day length required to induce and develop blooms Inflorescence form: Daisy Natural flowering season: Approximately the last two weeks in September Number of inflorescences per stem: Approximately 17 Inflorescence diameter: Approximately 3.1 cm Inflorescence depth: Approximately 2.2 cm Inflorescence longevity on plant: Approximately 24-29 days Persistence: Persistent

Ray florets.—Appearance: Matte Texture: Smooth Average Number per flower: Approximately 320 Shape: Oblanceolate Margin: Entire Apex: 3 acute lobes Length: Approximately 1.5 cm Width: Approximately 0.5 cm Color: upper surface at first opening: Near 53D upper surface at maturity: Near 51D at the tip; Near 36D at the base upper surface at fading: Near 65B at the tip; Near 155D at the base under surface at first opening: Near 54A under surface at maturity: Near 54C at the tip; Near 155D at the base under surface at fading: Near 56A at the tip; Near 155D at the base

Disc florets.—Appearance: Shiny Texture: Smooth Average number perflower: Approximately 100 Shape: Cylindric Apex: Obtuse Average Length: Approximately 0.7 cm Average Width: Approximately 0.1 cm Color: at first opening: Near 13A at maturity: Near 13B at fading: Near 15C

Peduncle.—Length: at terminal end (shortest): Approximately 4 cm at lateral end (longest): Approximately 7 cm Strength: Moderate Color: Near 138B Habit: Erect

Inflorescence bud.—Length: Approximately 0.6 cm Diameter: Approximately 0.6 cm Form: Globular Color: Near 51B

Sepals.—Appearance: Matte Texture: Lightly pubescent Number: Approximately 20 to 30 Shape: Ovate Margin: Entire Apex: Acute Color: upper side: Near 138A under side: Near 138A

Reproductive Organs

Ray Florets.—Pistillate: Number ofpistils per flower: 1 Stigma shape: 2 branched Stigma color: Near 16D Style color: Near 7A Style length: Approximately 0.4 cm

Disc florets.—Pistillate: Number ofpistils per flower: 1 Stigma shape: Cylindric Style length: Approximately 0.3 cm Staminate: Number ofstamens per flower: Approximately 5 Anther shape: Tubular Anther color: Near 12A Pollen color: Near 13A

Other Characteristics

Disease resistance.—Neither resistance nor susceptibility observed

What is claimed is:

1. A new and distinct variety of Chrysanthemum plant named ‘MOZA LOT 01’ as herein illustrated and described.
